(19) United States
(12) Patent Application Publication
(10) Pub. No.: US 2011/0129467 A1
(43) Pub. Date: Jun. 2, 2011

(54) THERAPEUTIC COMBINATION COMPRISING AN AURORA KINASE INHIBITOR AND ANTIPROLIFERATIVE AGENTS

(75) Inventors: Enrico Pesenti, Parabiago (MI) (IT); Dario Ballinari, San Donato Milanese (MI) (IT); Juergen Moll, Appiano Gentile (CO) (IT)

(73) Assignee: NERVIANO MEDICAL SCIENCES S.R.L., Nerviano (IT)

(21) Appl. No.: 13/054,868

(22) PCT Filed: Jul. 9, 2009
(86) PCT No.: PCT/EP2009/058779

§ 371 Date: Jan. 19, 2011

Related U.S. Application Data
(60) Provisional application No. 61/083,236, filed on Jul. 24, 2008.

(51) Int. Cl. A61K 39/395; A61K 31/69; A61K 31/496; A61P 35/00

(52) U.S. Cl. 424/133.1; 514/64; 514/254.06

(57) ABSTRACT

The present invention provides a combination comprising a compound 1 of formula (A) and one or more antineo-plastic agents selected from the group consisting of an antibody inhibiting a growth factor or a receptor of the growth factor, a proteasome inhibitor or a derivative or prodrug thereof, and a kinase inhibitor or a derivative or prodrug thereof useful in the treatment of tumors.

### FIELD OF THE INVENTION

The present invention relates in general to the field of cancer treatment and, more particularly, provides an anti-tumor composition comprising an aurora kinase inhibitor and an antibody inhibiting a growth factor and/or a proteasome inhibitor and/or a kinase inhibitor having a synergistic antiproliferative effect.

### BACKGROUND OF THE INVENTION

The malfunctioning of protein kinases (PKs) is the hallmark of numerous diseases. A large share of the oncogenes and proto-oncogenes involved in human cancers code for PKs. The enhanced activities of PKs are also implicated in many non-malignant diseases, such as benign prostate hyperplasia, familial adenomatosis, polyposis, neuro-fibromatosis, psoriasis, vascular smooth cell proliferation associated with atherosclerosis, pulmonary fibrosis, arthritis, glomerulonephritis and post-surgical stenosis and restenosis.

PKs are also implicated in inflammatory conditions and in the multiplication of viruses and parasites. PKs may also play a major role in the pathogenesis and development of neurodegenerative disorders.

For a general reference to PKs malfunctioning or disregulation see, for instance, Current Opinion in Chemical Biology, 1999, 3, 459-465.

Among the several protein kinases known in the art as being implicated in the growth of cancer cells are Aurora kinases, in particular Aurora-2.

Aurora-2 was found to be over-expressed in a number of different tumour types. Its gene locus maps at 20q13, a chromosomal region frequently amplified in many cancers, including breast [Cancer Res. 1999, 59(9), 2041-4] and colon cancers.

20q13 amplification correlates with poor prognosis in patients with node-negative breast cancer and increased Aurora-2 expression is indicative of poor prognosis and decreased survival time in bladder cancer patients [J. Natl. Cancer Inst., 2002, 94(17), 1320-9]. For a general reference to the Aurora-2 role in the abnormal centrosome function in cancer see also Molecular Cancer Therapeutics, 2003, 2, 589-595.

Several heterocyclic compounds are known in the art as protein kinase inhibitors. Among them, 3-carboxamido-pyrazoles and 3-ureido-pyrazoles and derivatives thereof, have been disclosed as protein kinase inhibitors in the international patent applications WO01/12189, WO12188, WO02/48114 and WO02/70515.

Fused bicyclic compounds comprising pyrazole moieties and possessing kinase inhibitory activity have been also disclosed in WO00/69846, WO02/12242, WO03/028720 and WO03/97610.

Specific tetrahydropyrrolo[3,4-c]pyrazole derivatives have been revealed to be potent ATP-competitive inhibitors of Aurora kinases (Fancelli, D., et al.: Journal of Medicinal Chemistry. 2005, vol. 48, no. 8, p. 3080-3084. PCT/WO 2005005427). Surprisingly, it has been found that the antineoplastic effect, i.e. especially the delay of progression or treatment of a proliferative disease, in particular the treatment of a tumour that is refractory to other chemotherapeutics known as antineoplastic agents, of the tetrahydropyrrolo[3,4-c]pyrazole derivatives above is greatly enhanced when they are administered in combination with certain antineoplastic agents. In particular, the antineoplastic effect of such combination is greater than the effects that can be achieved with either type of combination partner alone, i.e. greater than the effects of a monotherapy using only one of the combination partners.

The present invention thus provides new combinations of a tetrahydropyrrolo[3,4-c]pyrazole derivative with known pharmaceutical agents that are particularly suitable for the treatment of proliferative disorders, especially cancer. More specifically, the combinations of the present invention are very useful in therapy as antitumor agents and lack, in terms of both toxicity and side effects, the drawbacks associated with currently available antitumor drugs.

### SUMMARY OF THE INVENTION

The present invention provides a therapeutic combination comprising (a) Compound 1 of formula (A):

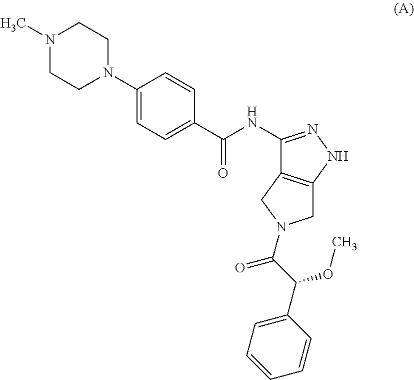

and (b) one or more antineoplastic agents selected from the group consisting of an antibody inhibiting a growth factor or a receptor of the growth factor, a proteasome inhibitor or a derivative or prodrug thereof, and a kinase inhibitor or a derivative or prodrug thereof, wherein the active ingredients are present in each case in free form or in the form of a pharmaceutically acceptable salt or any hydrate thereof.

The present invention also provides a combined preparation for simultaneous, separate or sequential use of the combinations as described above.

The present invention also provides a pharmaceutical composition comprising a combination as described above which is further admixed with a pharmaceutically acceptable carrier, diluent or excipient.

The present invention further provides a method of treating proliferative disorders by administering to a patient in need thereof a therapeutically effective amount of the combination as described above.

### DETAILED DESCRIPTION OF THE INVENTION

The present invention provides, in a first embodiment, a therapeutic combination comprising (a) Compound 1 of formula (A):

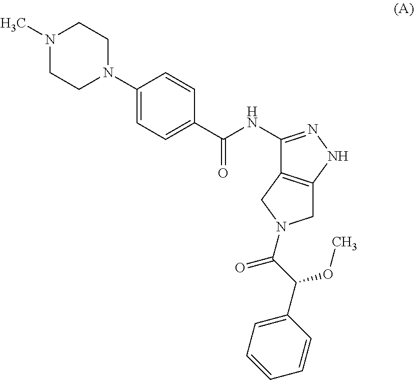

and (b) one or more antineoplastic agents selected from the group consisting of an antibody inhibiting a growth factor or a receptor of the growth factor, a proteasome inhibitor or a derivative or prodrug thereof, and a kinase inhibitor or a derivative or prodrug thereof, wherein the active ingredients are present in each case in free form or in the form of a pharmaceutically acceptable salt or any hydrate thereof.

In another embodiment the combination according to the invention is a combined preparation for simultaneous, separate or sequential use.

A further embodiment relates to the combination according to the invention in a method of treating or delaying the progression of a proliferative disorder, wherein the method comprises the simultaneous, sequential or separate administration to a patient in need thereof of the therapeutic combination.

In a still further embodiment the invention provides a pharmaceutical composition comprising a combination according to the invention admixed with a pharmaceutically acceptable carrier, diluent or excipient.

A still further embodiment relates to the use of compound 1 of formula (A) as defined above in the preparation of a medicament for the treatment of a proliferative disorder, wherein said treatment comprises simultaneously, sequentially or separately administering compound 1 of formula (A) as defined above and one or more antineoplastic agents selected from an antibody inhibiting a growth factor or a receptor of the growth factor, a proteasome inhibitor or a derivative or prodrug thereof, and a kinase inhibitor or a derivative or prodrug thereof, to a patient in need thereof.

Compound 1 of formula (A) has the chemical name N-{5-[(2R)-2-methoxy-2-phenylethanoyl]-1,4,5,6-tetrahydropyrrolo[3,4-c] pyrazol-3-yl}-4-(4-methylpiperazin-1-yl)benzamide. It can be prepared as described in WO 2005/005427 (incorporated herein by reference), is endowed with protein kinase inhibitory activity and is thus useful in therapy as antitumor agent.

Pharmaceutically acceptable salts of the compound of formula (A) include the acid addition salts with inorganic or organic acids, e.g., nitric, hydrochloric, hydrobromic, sulphuric, perchloric, phosphoric, acetic, trifluoroacetic, propionic, glycolic, lactic, oxalic, malonic, malic, maleic, mesylate, tartaric, citric, benzoic, cinnamic, mandelic, methanesulphonic, isethionic and salicylic acid and the like.

According to a preferred embodiment of the invention, the antibody inhibiting growth factor or a receptor of the growth factor is selected from the group consisting of bevacizumab (antibody to vascular endothelial growth factor), cetuximab, panitumumab, matuzumab, nimotuzumab (antibodies to epidermal growth factor receptor), trastuzumab and pertuzumab (antibodies to ErbB2).

According to a more preferred embodiment of the invention, the antibody inhibiting growth factor or a receptor of the growth factor is bevacizumab. Bevacizumab is marketed as Avastin®.

Proteasome inhibitors are drugs that block the action of proteasomes, cellular complexes that break down proteins. According to a preferred embodiment of the invention the proteasome inhibitor is preferably bortezomib. Bortezomib, marketed as Velcade® by Millennium Pharmaceuticals, was the first therapeutic proteasome inhibitor to be tested in humans. It is approved in the U.S. for treating relapsed multiple myeloma and mantle cell lymphoma.

According to a preferred embodiment of the invention, the kinase inhibitor is flavopiridol or dasatinib.

In the present invention, each of the active ingredients of the combination is provided in an amount effective to produce a synergistic antineoplastic effect.

The present invention also provides a method for lowering the side effects caused by antineoplastic therapy with an antineoplastic agent in mammals, including humans, in need thereof, the method comprising administering to said mammal a combined preparation comprising the compound of formula (A) as defined above and one or more antineoplastic agents selected from the group consisting of an antibody inhibiting a growth factor or a receptor of the growth factor, a proteasome inhibitor or a derivative or prodrug thereof, and a kinase inhibitor or a derivative or prodrug thereof, in amounts effective to produce a synergistic antineoplastic effect.

By the term “a synergistic antineoplastic effect” as used herein is meant the inhibition of the growth of the tumor, preferably the complete regression of the tumor, by administering an effective amount of the combination of compound 1 of formula (A) as defined above and one or more antineoplastic agents selected from the group consisting of an antibody inhibiting a growth factor or a receptor of the growth factor, a proteasome inhibitor or a derivative or prodrug thereof, and a kinase inhibitor or a derivative or prodrug thereof to mammals, including humans.

The term “combined preparation” as used herein defines especially a “kit of parts” in the sense that the combination components (a) and (b) as defined above can be dosed independently or by use of different fixed combinations with distinguished amounts of the combination components (a) and (b), i.e. simultaneously or at different time points. The elements of the kit of parts can then, e.g., be administered simultaneously or chronologically staggered, that is at different time points and with equal or different time intervals for any part of the kit of parts. Most preferably, the time intervals are chosen such that the effect on the treated disease in the combined use of the parts is greater than the effect which would be obtained by use of only any one of the combination components (a) and (b). The ratio of the total amounts of the combination component (a) to the combination component (b) to be administered in the combined preparation can be varied, e.g. in order to cope with the needs of a patient sub-population to be treated or the needs of the single patient which different needs can be due to the particular disease, age, sex, body weight, etc. of the patients. Preferably, there is at least one beneficial effect, e.g., a mutual enhancing of the effect of the combination components (a) and (b), in particular a synergism, e.g. a more than additive effect, additional advantageous effects, less side effects, less toxicity, or a combined therapeutic effect in a non-effective dosage of one or both of the combination components (a) and (b), and very preferably a strong synergism of the combination components (a) and (b).

By the term “administered” or “administering” as used herein is meant parenteral and/or oral administration. By “parenteral” is meant intravenous, subcutaneous and intramuscular administration.

In the method of the subject invention, for the administration of compound 1 of formula (A), the course of therapy generally employed is in the range from 100 mg/m²/day to 1500 mg/m²/day of body surface area for up to 21 consecutive days. More preferably, the course therapy employed is from about 150 mg/m²/day to about 350 mg/m²/day of body surface area for up to 21 consecutive days.

In a particularly preferred regimen, the compound of formula (A) is administered in a dose of 190 or 250 mg/m²/day of body surface area for three hours infusion on day 1 and 8 of a three weeks cycle. Other possible therapeutic schedules are disclosed, for example, in WO2008/052931 published May 8, 2008 (incorporated herein by reference).

Compound 1 of formula (A) can be administered in a variety of dosage forms, e.g., orally, in the form of tablets, capsules, sugar or film coated tablets, liquid solutions or suspensions; rectally in the form of suppositories; parenterally, e.g., intramuscularly, or through intravenous and/or intrathecal and/or intraspinal injection or infusion.

In the method of the subject invention, for the administration of the proteasome inhibitor, preferably bortezomib, the course of therapy generally employed is from about 50 mg/m² to 100 mg/m² every three weeks or from 30 mg/m² weekly.

For the administration of an antibody inhibiting a growth factor, preferably bevacizumab, the course of therapy generally employed may be from 0.1 mg/kg to 100 mg/kg. More preferably the course of therapy employed is from 1 mg/kg to 20 mg/kg on day 1 of a three weeks cycle.

For the administration of a kinase inhibitor, preferably flavopiridol, the course of therapy generally employed may be from 0.1 mg/kg to 100 mg/kg. More preferably the course of therapy employed is from 1 mg/kg to 20 mg/kg.

The anti-neoplastic therapy of the present invention is in particular suitable for treating all forms of cancer including, but not limited to: carcinoma such as bladder, breast, colon, kidney, liver, lung, including small cell lung cancer, esophagus, gall- bladder, ovary, pancreas, stomach, cervix, thyroid, prostate, and skin, including squamous cell carcinoma; haematopoietic tumors of lymphoid lineage including leukaemia, acute lymphocytic leukaemia, acute lymphoblastic leukaemia, B-cell lymphoma, T-cell-lymphoma, Hodgkin's lymphoma, non-Hodgkin's lymphoma, mantle cell lymphoma, hairy cell lymphoma and Burkett's lymphoma; haematopoietic tumors of myeloid lineage, including acute and chronic myelogenous leukemias, myelodysplastic syndrome and promyelocytic leukaemia; multiple myeloma; tumors of mesenchymal origin, including fibrosarcoma and rhabdomyosarcoma; tumors of the central and peripheral nervous system, including astrocytoma neuroblastoma, glioma and schwannomas; other tumors, including melanoma, seminoma, teratocarcinoma, osteosarcoma, xeroderma pigmentosum, keratoxanthoma, thyroid follicular cancer and Kaposi's sarcoma.

As stated above, the effect of the combination of the invention is significantly increased without a parallel increased toxicity. In other words, the combined therapy of the present invention enhances the antitumoral effects of the component (a) and/or of component (b) of the combination of the invention and thus yields the most effective and less toxic treatment for tumors.

Pharmaceutical compositions according to the invention are useful in anticancer therapy.

The present invention further provides a commercial package comprising, in a suitable container means, (a) a compound of formula (A) as defined above, and (b) one or more antineoplastic agents selected from the group consisting of an antibody inhibiting a growth factor or a receptor of the growth factor, a proteasome inhibitor or a derivative or prodrug thereof, and a kinase inhibitor or a derivative or prodrug thereof, wherein the active ingredients are present in each case in free form or in the form of a pharmaceutically acceptable salt or any hydrate thereof, together with instructions for simultaneous, separate or sequential use thereof.

In a package according to the invention each of component (a) and (b) are present within a single container means or within distinct container means.

Another embodiment of the present invention is a commercial package comprising a pharmaceutical composition or product as described above.

Due to the key role of the aurora kinase proteins in the regulation of cellular mitosis and proliferation, the combinations of the present invention are also useful in the treatment of a variety of cell proliferative disorders such as, for example, benign prostate hyperplasia, familial adenomatosis, polyposis, neurofibromatosis, psoriasis, vascular smooth cell proliferation associated with atherosclerosis, pulmonary fibrosis, arthritis, glomerulonephritis and post-surgical stenosis and restenosis.

The combinations of this invention, as modulators of apoptosis, may also be useful in the treatment of cancer, viral infections, prevention of AIDS development in HIV-infected individuals, autoimmune diseases and neurodegenerative disorders. The activities of the combination of the present invention are shown for instance by the following in vitro and in vivo tests, which are intended to illustrate but not to limit the present invention.

### EXAMPLE 1

### Materials and Methods For In Vitro Cytotoxic Activity Testing

Exponentially growing human colon carcinoma (HCT-116) cell lines were seeded and incubated at 37° C. in a humidified 5% CO₂ atmosphere. Drugs were added to the experimental culture, and incubations were carried out at 37° C. for 72 hours in the dark. Scalar doses of Compound 1 of formula (A) as defined above and antineoplastic agent were added to the medium 24 hours after seeding. Sequential administration schedule was tested (Compound 1 administered 24 hours after the other agent). Drug solutions were prepared immediately before use. At the end of treatment, cell proliferation was determined by an intracellular adenosine triphosphate monitoring system (CellTiterGlo-Promega) using the Envision (PerkinElmer) as reader. Inhibitory activity was evaluated comparing treated versus control data using Assay Explorer (MDL) program. The dose inhibiting 50% of cell growth was calculated using sigmoidal interpolation curve. Combination indices (C.I.) were calculated using a computer program for multiple drug effect analysis based on the equation of Chou-Talalay (Adv Enzyme Regul 1984; 22:27-55) for mutually nonexclusive drugs, where a C.I.<1 indicates a more than additive effect (C.I.>3 indicates strong antagonism; 1.3<C.I.<3, antagonism; 0.8<C.I.<1.3, additivity; 0.3<C.I.<0.8, synergism; C.I.<0.3, strong synergism).

### Example 2

### In Vitro Cytotoxic Activity of the Combination With Flavopiridol

The results shown in Table 1 indicate that on human colon carcinoma HCT-116 cell line the administration of Compound 1 in combination with flavopiridol resulted in a synergistic antitumor effect.

TABLE 1

| | Drug | | | | |
|---|---|---|---|---|---|
| | Compound 1 | Flavopiridol | | | Effect of |
| Cell line | μM | μM | Schedule | C. I. | combination |
| HCT-116 | 0.5 | 0.62 | Sequential | 0.63 | Synergism |
| | 0.25 | 0.62 | | 0.63 | Synergism |

### Example 3

### In Vivo Antitumor Efficacy of the Combination With the Monoclonal Antibody Bevacizumab

Balb, Nu\Nu male mice, from Harlan (Italy), were maintained in cages with paper filter cover, food and bedding sterilized and water acidified. 2.5×10⁶ DU145 prostate carcinoma cells (from the American Type Culture Collection) were injected subcutaneously in athymic mice. This tumor model was selected because it was previously demonstrated that Bevacizumab inhibits angiogenesis and growth of the model in vivo (see for reference The Prostate 36: 1-10, 1998). The treatment started at day 9 after cell injection, when tumors were palpable with a tumor weight mean for all the groups of 0.15 g. Bevacizumab was prepared immediately before treatment, while Compound 1 was prepared daily, on the basis of known stability of the compound.

Compound 1 was administrated by intraperitoneal route in a volume of 10 ml/kg at the dose of 15 mg/kg twice a day (BID) for 9 days from day 9 to day 17. Bevacizumab was administered intraperitoneally in a volume of 10 ml/kg at the dose of 20 mg/kg on days 9, 13, 17 from the days of tumor cells injection. When combined, Compound 1 was administered at days 9, 10, 11, 12, 13, 14, 15, 16, 17, 18 and 19, bevacizumab was administered at days 9, 13 and 17 immediately after Compound 1 administration. Tumor growth and body weight were measured every 3 days. Tumor growth was assessed by caliper. The two diameters were recorded and the tumor weight was calculated according the following formula: length (mm)×width²/2 .The effect of the antitumor treatment was evaluated as the delay in the onset of an exponential growth of the tumor (see for references, Anticancer drugs 7:437-60, 1996). This delay (T-C value) was defined as the difference of time (in days) required for the treatment group (T) and the control group(C) tumors to reach a predetermined size (1 g). Toxicity was evaluated on the basis of body weight reduction. The results are reported in Table 2 below. Compound 1 combined with bevacizumab produced a clear additive/synergistic effect: the T-C observed when Compound 1 was combined with bevacizumab was superior to the expected by the simple addition of T-C obtained by the single treatments. No toxicity was observed in any of the treatment group.

TABLE 2

| | Time to reach | | |
|---|---|---|---|
| Treatment | 1 g (days) | T-C (days) | Toxicity |
| Compound 1 | 23.9 | 8.5 | 0/8 |
| 15 mg/kg* | | | |
| Bevacizumab | 18.5 | 3.1 | 0/8 |
| 20 mg/kg** | | | |
| Bevacizumab | 28.2 | 12.8 | 0/8 |
| 20 mg/kg + | | | |
| Compound 1 | | | |
| 15 mg/kg*** | | | |

*Treatments made intraperitoneally twice at days 9, 10, 11, 12, 13, 14, 15, 16, 17, 18 and 19
**Treatments made intraperitoneally at days 9, 13 and 17
***Days 9, 13 and 17 bevacizumab treatments; days 9, 10, 11, 12, 13, 14, 15, 16, 17, 18 and 19 Compound 1 treatments

### Example 4

### In Vivo Antitumor Efficacy of the Combination With Bortezomib

SCID male mice, from Harlan (Italy), were maintained in cages with paper filter cover, food and bedding sterilized and water acidified. 5×10⁶ HL-60 human acute myeloid leukaemia cells (from the American Type Culture Collection) were injected subcutaneously in SCID mice. This tumor model was selected because it was previously demonstrated that Compound 1 inhibits growth of the model in vivo and also on the basis of use of this model as representative of haematological malignancy.

The treatment started 12 days later tumor cells injection when tumors were palpable. Bortezomib was prepared immediately before treatment, Compound 1 was prepared daily, on the basis of known stability of the compound.

Compound 1 was administered by intraperitoneal route in a volume of 10 ml/kg at the doses of 15 mg/kg twice a day (BID) for 12 days (days 12 to 23). Bortezomib was administered by intravenous route in a volume of 10 ml/kg at the dose of 0.5 mg/kg on days 12, 16, 20 and 24 from the days of cell injection, and at the dose of 1 mg/kg on days 12, 16, 20 and 24 from the days of cell injection. When combined, Compound 1 was administered in the interval between the bortezomib treatments at days 13, 14, 15, 17, 18, 19, 21, 22, 23, 25, 26 and 27. Tumor growth and body weight were measured every 3 days. Tumor growth was assessed by caliper. The two diameters were recorded and the tumor weight was calculated according the following formula: length (mm)×width²/2. The effect of the antitumor treatment was evaluated as the delay in the onset of an exponential growth of the tumor (see for references, Anticancer drugs 7:437-60, 1996). This delay (T-C value) was defined as the difference of time (in days) required for the treatment group (T) and the control group (C) tumors to reach a predetermined size (1 g). Toxicity was evaluated on the basis of body weigh reduction. The results are reported in Table 3 below. Compound 1 combined with 0.5 mg/kg of bortezomib produced a clear additive/synergistic effect: the T-C observed when Compound 1 was combined with 0.5 mg/kg of bortezomib was superior to the expected by the simple addition of T-C obtained by the single treatments. No toxicity was observed in the treatment groups. Compound 1 combined with 1 mg/kg of bortezomib produced a strong synergistic effect: the T-C observed when Compound 1 was combined with 1 mg/kg of bortezomib (35.7) was clearly superior to the expected by the simple addition of T-C obtained by the single treatments (17.3). Some signs of toxicity were observed in the treatment groups.

TABLE 3

| | Time to reach | | |
|---|---|---|---|
| Treatment | 1 g (days) | T-C (days) | Toxicity |
| Compound 1 | 24.7 | 9.1 | 0/7 |
| 15 mg/kg* | | | |
| Bortezomib | 19 | 3.4 | 0/7 |
| 0.5 mg/kg** | | | |
| Bortezomib | 23.8 | 8.2 | 0/7 |
| 1 mg/kg** | | | |
| Bortezomib | 29.2 | 13.5 | 0/7 |
| 0.5 mg/kg + | | | |
| Compound 1 | | | |
| 15 mg/kg*** | | | |
| Bortezomib | 51.4 | 35.7 | 3/7 |
| 1 mg/kg + | | | |
| Compound 1 | | | |
| 15 mg/kg*** | | | |

*Treatments made intraperitoneally twice at days 12, 13, 14, 15, 16, 17, 18, 19, 20, 21, 22, 23.
**Treatments made by intravenous route at days 12, 16, 20, 24
***Days 12, 16, 20 24 bortezomib treatments, 13, 14, 15, 17,18, 19, 21, 22, 23, 25, 26 and 27 Compound 1 treatments.

1-11. (canceled)

12. A therapeutic combination comprising (a) Compound 1 of formula (A): 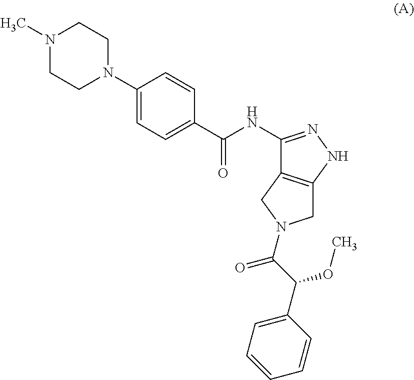
and (b) one or more antineoplastic agents selected from the group consisting of bevacizumab, bortezomib and flavopiridol, wherein the active ingredients of the combination are present in each case in free form or in the form of a pharmaceutically acceptable salt or any hydrate thereof.

13. The combination according to claim 12 which is a combined preparation for simultaneous, separate or sequential use.

14. A pharmaceutical composition comprising the combination according to claim 12 admixed with a pharmaceutically acceptable carrier, diluent or excipient.

15. A method of treating or delaying the progression of a proliferative disorder in a patient in need thereof comprising administering to said patient a therapeutically effective amount of the therapeutic combination of claim 12.

16. A method for lowering the side effects caused by antineoplastic therapy with an antineoplastic agent in mammals in need thereof comprising administering to said mammal a therapeutically effective amount of the therapeutic combination of claim 12.

17. A commercial package comprising, in a suitable container means, (a) compound 1 of formula (A) as defined in claim 12, and (b) one or more antineoplastic agents selected from the group consisting of bevacizumab, bortezomib and flavopiridol, wherein the active ingredients are present in each case in free form or in the form of a pharmaceutically acceptable salt or any hydrate thereof, together with instructions for simultaneous, separate or sequential use thereof.
